(19) United States
(12) Patent Application Publication
(10) Pub. No.: US 2023/0131888 A1
(43) Pub. Date: Apr. 27, 2023

(54) METHOD OF ANALYZING QUALITY OF OSTEOCHONDRAL GRAFT THROUGH MICRO-CT ASSAY

(71) Applicant: ATEMS CO., LTD., Seoul (KR)

(72) Inventors: Byoung-Hyun MIN, Seoul (KR); Young-Jun JIN, Suwon-si, (KR)

(73) Assignee: ATEMS CO., LTD., Seoul (KR)

(21) Appl. No.: 18/049,848

(22) Filed: Oct. 26, 2022

(30) Foreign Application Priority Data
Oct. 26, 2021 (KR) 10-2021-0143787

(51) Int. Cl. G01N 33/50; G01N 23/046; G01N 23/083

(52) U.S. Cl. CPC G01N 33/5091; G01N 23/046; G01N 23/083; G01N 2223/04; G01N 2223/419

(57) ABSTRACT

Proposed is a non-destructive osteochondral graft quality analysis method using a Micro-CT assay, rather than using an existing biochemical assay involving destructive pretreatment of osteochondral tissue samples. Since the GAG content of the osteochondral graft can be measured by the non-destructive method through the Micro-CT assay, the quality analysis of the osteochondral graft provided by a donor can be easily performed, and the therapeutic effect of the osteochondral transplantation can be improved by enabling the use of a graft with a GAG content of 70% or more.

### CROSS REFERENCE TO RELATED APPLICATION

The present application claims priority to Korean Patent Application No. 10-2021-0143787, filed Oct. 26, 2021, the entire contents of which is incorporated herein for all purposes by this reference.

### BACKGROUND OF THE INVENTION

### 1. Field of the Invention

The present disclosure relates to a non-destructive quality analysis method for osteochondral grafts using a Micro-CT assay, rather than a conventional biochemical assay involving destructive pretreatment of osteochondral tissue samples.

### 2. Description of the Related Art

Cartilage damage can be caused by degenerative changes in the joint, overuse of the joint, or damage to the joint by trauma and can easily lead to osteoarthritis because of the limited regenerative capacity of the cartilage.

Allogeneic osteochondral transplantation is one of the representative treatment methods for healing a wide range of articular cartilage defects that occur as a result of extensive cartilage damage. Allogeneic osteochondral transplantation has a superior therapeutic effect on a wider range of cartilage defects to conventional microfracture and autologous chondrocyte transplantation that have been commonly implemented.

However, osteochondral allografts vary in quality depending on donors' conditions, and when inappropriate osteochondral grafts are transplanted, the grafts are not easily fused with host tissue, and collapse of the transplanted graft occurs due to bone tissue necrosis. Therefore, the pre-transplantation quality analysis of a graft provided by a donor is directly related to the success of a transplantation surgery.

On the other hand, the major components of cartilage tissue are chondrocytes and cartilage extracellular matrix (ECM). In connection with chondrocytes, for successful allogeneic osteochondral transplantation, cell viability in the transplanted graft may be a crucial factor. According to the results of long-term follow-up studies for more than 10 years on osteochondral transplantation, it is reported that significant clinical therapeutic efficacy is observed when the cell viability of the osteochondral graft is 70% or more.

On the other hand, in the study of allogeneic osteochondral transplantation involving ECM, it has been confirmed that when the calf cartilage tissue was treated with a decomposing enzyme so that glycosaminoglycans (GAGs), the main component of cartilage tissue ECM, were decomposed, and physical external stress is applied thereto, the chondrocytes in a site where the GAGs were degenerated were significantly killed. Accordingly, the GAG content of the cartilage ECM plays a vital role in chondrocyte viability of cartilage tissue.

Currently, biochemical assays are commonly used to measure the GAG content of cartilage tissue, and the methods spectroscopically measure the GAG content through disruption and water-solubilization of tissue.

Therefore, biochemical assays involving destructive pretreatment of tissue samples are not suitable for quality analysis of osteochondral tissue grafts provided by donors. Therefore, a test method capable of non-destructively analyzing the GAG content is required.

### LITERATURE OF RELATED ART

### Patent Literature

(Patent Literature 1) Korean Patent No. 10-2278665 (Jul. 12, 2021)

### SUMMARY OF THE INVENTION

It is an objective of the present disclosure to provide a non-destructive quality analysis method for osteochondral grafts using a Micro-CT assay, rather than a biochemical assay involving destructive pretreatment of osteochondral tissue samples.

The present disclosure provides a method of analyzing the quality of an osteochondral graft so that osteochondral tissue isolated from an individual can be used as a transplantation graft. The method includes analyzing the glycosaminoglycan (GAG) content of the osteochondral tissue.

According to the present disclosure, osteochondral tissue is treated with a contrast medium, the absorbance value of the osteochondral tissue is measured through a Micro-CT assay, the GAG content of the same kind of osteochondral tissue is measured through a biochemical assay, and a formula that reflects the correlation of the two numerical values is formulated. With the use of the formula and the Micro-CT value, it is possible to non-destructively measure the GAG content of the osteochondral graft. Therefore, the quality of a donor's osteochondral graft can be easily analyzed, and thus the therapeutic efficacy of osteochondral transplantation can be improved with the use of a graft with a GAG content of 70% or more.

### BRIEF DESCRIPTION OF THE DRAWINGS

FIG. 1 is a schematic view according to one embodiment of the present disclosure, and illustrates showing: a process of treating human osteochondral tissue with a contrast medium (IOBRIX 300) and performing a Micro-CT assay and a biochemical assay to measure the GAG content of the tissue; a process of reducing the GAG content of a rabbit allogeneic osteochondral graft with chondroitinase ABC, which is a GAG-specific degrading enzyme and of performing a Micro-CT assay, a biochemical assay, and a histological assay to measure the GAG content; and a process of evaluating the therapeutic efficacy after transplanting grafts that are regulated to have different GAG content levels according to the chondroitinase ABC treatment time.

FIG. 2 shows a comparison of Micro-CT absorbance values and Hounsfield Units (HU) according to contrast treatment time.

FIGS. 3A to 3B show the changes in GAG content according to knee joint femoral cartilage sites for each age in the 30s, 40s, 50s and 60s, with human osteochondral tissue in the ages of 10s and 20s set as normal human osteochondral tissue, and FIG. 3C shows the result of comparison (expressed in percentages) between the GAG content of the osteochondral cartilage for each age compared to the GAG content of normal osteochondral cartilage in teens and 20s.

FIG. 4 is a graphical match of Micro-CT analysis values according to knee joint femoral cartilage sites of human osteochondral tissue for each age.

FIG. 5 is a graphical match of the results of biochemical assay and Micro-CT assay of human osteochondral tissue.

FIGS. 6A and 6B show the results of histological assay and Micro-CT assay of human osteochondral tissue for each group and specifically shows optical images and numerical values of optical densities, in the cases of Safranin-O staining (FIG. 6A) and Micro-CT assay (FIG. 6B).

FIGS. 7A. 7B, and 7C show the GAG contents of osteochondral grafts in a rabbit allogeneic osteochondral grafting model in which the GAG contents of rabbit osteochondral grafts are reduced according to chondroitinase treatment time. The GAG contents are measured through biochemical assay, Micro-CT assay, and histological assay according to chondroitinase treatment time, and the measured GAG contents are compared to the GAG content of a normal group and expressed in percentages (see FIGS. 7A and 7B). The GAG contents of rabbit osteochondral grafts are determined through Micro-CT assay and biochemical assay, and the correlation therebetween is expressed as a formula (FIG. 7C).

FIG. 8 shows the results of allogeneic rabbit osteochondral grafting after the GAG contents of rabbit osteochondral grafts which are controlled by the chondroitinase treatment are analyzed by Micro-CT assay.

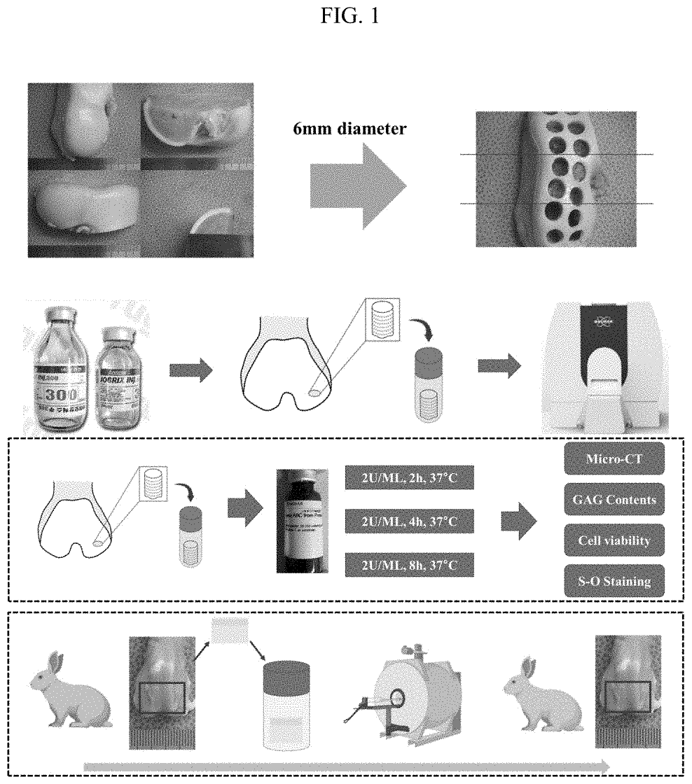

### DESCRIPTION OF THE PREFERRED EMBODIMENTS

Hereinafter, the present disclosure will be described in detail.

In order to overcome the problem of existing biochemical assays involving destructive pre-treatment for quality analysis of osteochondral tissue grafts, the inventors have made an effort and completed the invention relating to a method of non-destructively analyzing a GAG content by using a Micro-CT assay.

The present disclosure provides a method of analyzing the quality of an osteochondral graft so that osteochondral tissue isolated from an individual can be used as a transplantation graft. The method includes analyzing the glycosaminoglycan (GAG) content in the osteochondral tissue through a Micro-CT assay.

The osteochondral graft may be prepared by a conventional method of preparing a graft material by harvesting osteochondral tissue samples isolated from a subject and shaping and lyophilizing the collected tissue samples.

The micro-CT assay may be performed by diluting an iodine-based contrast medium with a phosphate buffer solution to a concentration of 50% (v/v) to 60% (v/v) and treating the tissue samples with the resulting solution for 12 to 16 hours. In this case, when the concentration of the contrast medium is out of the described range, the osmosis rate of the contrast medium decreases, and the contrast medium treatment time increases. When the contrast medium treatment time is excessively short or long, partial osmosis of the contrast medium occurs in the tissue or the contrast medium treatment time is long, resulting in a problem of affecting tissue quality.

The analysis method determines a GAG content by converting a CT attenuation coefficient (Hounsfield unit, HU) value of the Micro-CT assay and uses the obtained value as a criterion of osteochondral graft quality.

The iodine-based contrast medium may be selected from the group consisting of, but is not limited to, Iohexol, Nucleosabrix, and Ioxaglate.

With the use of the analysis method according to the present disclosure, it is possible to determine a graft with a GAG content of 70% or more to be a suitable graft for osteochondral transplantation because such a graft exhibits a good transplantation rate and good therapeutic efficacy.

The osteochondral tissue may be, but is not limited to, human- or rabbit-derived osteochondral tissue, and it may be allogeneic osteochondral tissue.

In particular, according to the present disclosure, after the osteochondral tissue was treated with a contrast medium, the absorbance level of the osteochondral tissue was measured through Micro-CT assay, and the GAG content of the osteochondral tissue was measured through biochemical assay. Then, the correlation of the two values was formulated into a formula. By using the correlation formula, the GAG content of an osteochondral graft can be measured by inputting the Micro-CT measurement value into the formula without using the conventional biochemical assay.

That is, the present disclosure provides an osteochondral graft quality analysis method for determining the GAC content of an osteochondral graft by using a formula that reflects the correlation between the glycosaminoglycan (GAG) content and the CT attenuation coefficient (Hounsfield unit, HU) of Micro-CT.

In one embodiment of the present disclosure, when using human osteochondral tissue, the quality analysis of the osteochondral graft can be performed by calculating the GAG content using the following Formula 1:

y=−4.76x+2000.6 [Formula 1]

In Equation 1, x is the osteochondral GAG content (μg/mg) based on dry weight, and y is the CT attenuation coefficient (HU).

In another embodiment of the present disclosure, when using rabbit osteochondral tissue, the quality analysis of the osteochondral graft can be performed by calculating the GAG content by using the following Formula 2:

y=−7.1285x+1497.1 [Formula 2]

In Equation 2, x is the osteochondral GAG content (μg/mg) based on dry weight, and y is the CT attenuation coefficient (HU).

In the present disclosure, differences in GAG content of osteochondral tissue according to human age were analyzed through micro-CT assay and biochemical assay. In addition, the present disclosure presents a standard of GAG content representing a significant healing result obtained by osteochondral graft transplantation by transplanting several osteochondral grafts with respectively different GAG content levels using a rabbit allogeneic osteochondral transplantation model.

As described above, the present disclosures invention proposes a method for measuring the GAG content of an allogeneic osteochondral graft through non-destructive Micro-CT assay and presents criteria for determining the quality of the allogeneic osteochondral graft implants.

Hereinafter, in order to help the understanding of the present disclosure, examples will be described in detail. However, the examples described below are provided only for illustrative purposes, and thus the scope of the present disclosure is not limited by the examples described below. The examples are provided to aid those skilled in the art to more easily understand the invention of the present disclosure.

### Example 1: Optimize Contrast Treatment Conditions for Human Osteochondral Tissue

Human cartilage tissue was acquired from the Life Link Tissue Bank in the United States, and one half-femoral knee joint (hemi-condyle) was donated per donor for experiments. In order to evaluate the change in the GAGs composition in cartilage tissue according to age, test groups for ages of 10s, 20s, 30s, 40s, 50s, and 60s were constructed. 3 samples were collected from each age group, and the test was conducted according to the rules of the institutional research ethics review committee (research approval number: AJIRB-BMR-SMP-19-366) of Ajou University Hospital. Osteochondral tissue from each cartilage sample was collected using a 6-mm biopsy punch. A cartilage contrast medium (Iobrix 300, Taejun Pharmaceutical Company) was diluted to different concentrations using phosphate buffered saline (PBS). Samples taken from the same cartilage tissue were treated with the previously prepared contrast mediums with different concentrations that are 100% (v/v), 90% (v/v) %, 80% (v/v), 70% (v/v), 60%% (v/v), and 50%% (v/v). Micro-CT assay was performed at different treatment times: 1 hour (1H), 2 hours (2H), 4 hours (4H), 6 hours (4H), 8 hours (8H), 10 hours (10H), 12 hours (12H), 14 hours (14H), 16 hours (16H), 20 hours (20H), 24 hours (24H). Finally, the contrast medium treatment conditions were optimized on the basis of the Micro-CT assay results according to each contrast medium concentration and each contrast medium treatment time.

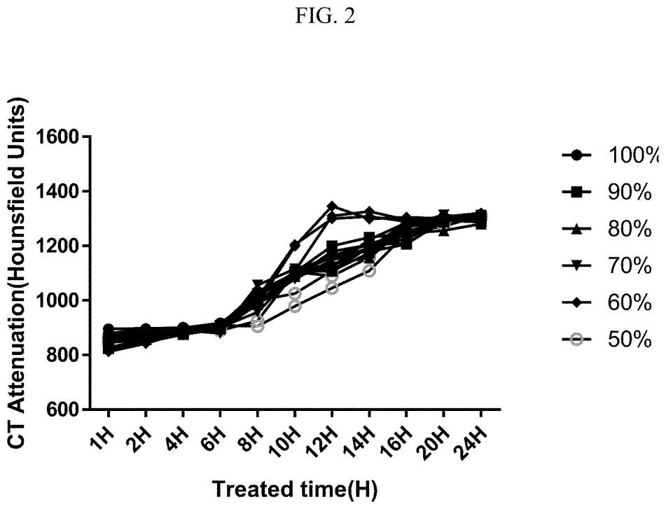

CT attenuation coefficients (HUs) were analyzed according to each contrast medium concentration and each contrast medium treatment time. As a result, as shown in FIG. 2, the HU values peaked at 12H in the 60% (v/v) contrast treatment group. In the group, the time taken to reach the maximum HU value was shorter than those of the higher concentration contrast treatment groups. Therefore, in the present disclosure, the optimized contrast concentration is determined to be 60% (v/v) and the optimized treatment time is determined to be 12 hours.

Hereinafter, the analysis was performed according to the optimized contrast concentration and the optimized contrast treatment time.

### Example 2: Analysis of GAG Content of Human Osteochondral Grafts According to Age

Cartilage tissue samples at different anatomical sites (four sites: trochlea, anterior, distal, and posterior) of the femoral condyle for each age group, which were provided by the tissue bank, were randomly selected. The same contrast medium as in Example 1 was diluted to 60% (v/v), and the tissue samples were treated for 12 hours with the diluted contrast medium. Next, the HU values were measured through Micro-CT assay. Cartilage tissue samples at the same sites were then measured for the GAG content through biochemical assay.

More specifically, each 6-mm sample taken from the cartilage tissue was placed in a 1.5-ml tube, 1.5 ml of the contrast medium was added, and the sample was treated for 12 hours. The contrast-treated tissues were removed from the tubes, wiped with PBS-soaked gauze, and seated on Micro-CT instruments to analyze HU values. In the biochemical assay, each of the cartilage tissues was cut with a surgical blade to have a thickness of 1 mm and lyophilized. Next, the weight of the lyophilized cartilage tissue was measured and mixed with a Papain (Sigma-Aldrich) solution at a ratio of 1 mg/ml and dissolved in a 60° C. oven for 24 hours. The cartilage tissue solution resulting from the dissolution was analyzed for the GAG content per unit weight the cartilage tissue, using the Glycosaminoglycan Assay Blyscan™ (1,9-dimethylmethylene blue) assay kit.

In addition, after measuring the GAG contents for the ages of 10s, 20s, 30s, 40s, 50s, and 60s through Micro-CT assay and biochemical assay, the tissues of the ages of 10s and 20s were set as normal cartilage tissues, and the GAG content in the other groups was quantified as a percentage compared to the GAS content of the normal groups.

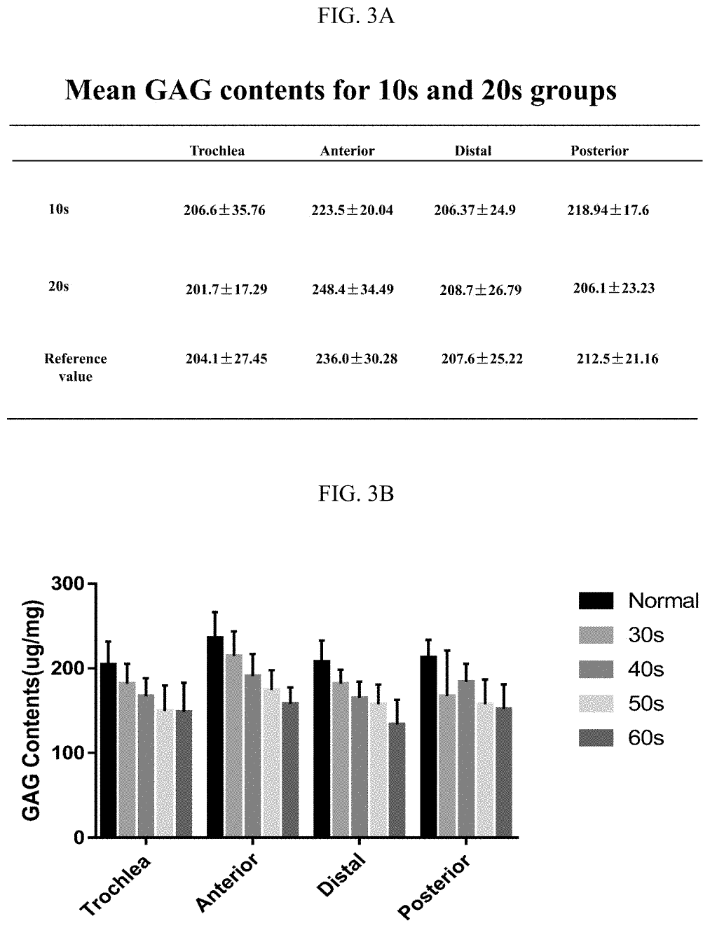

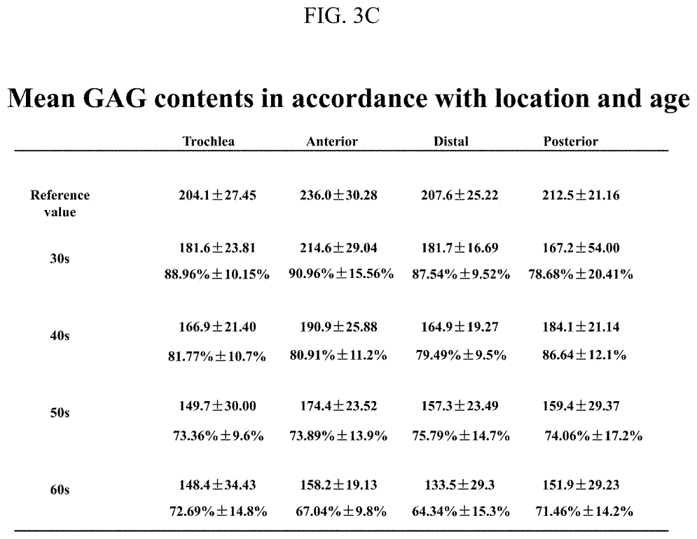

As a result, as illustrated in FIGS. 3A and 3B, it was confirmed through the biochemical assay that the GAG content varied depending on the knee joint femoral cartilage site in each age group, and as illustrated in FIG. 3C, it was confirmed that the GAG content decreased with age, when compared with the GAG content of the ages of 10s and 20s.

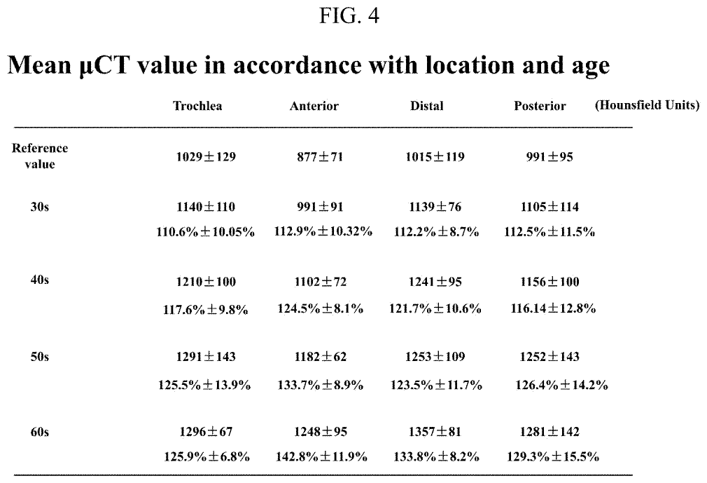

In FIG. 4, it was confirmed through the Micro-CT assay that the GAG content varied depending on the knee joint femoral cartilage site in each age group.

### Example 3: Derive Correlation in GAG Content Between Micro-CT Assay and Biochemical Assay

On the basis of the results of the GAG concentration measured through the biochemical assay in Example 2 and the results of the absorbance value measured for the same site through the Micro-CT assay, the correlation between the results of the two assays was derived and formulated into a formula.

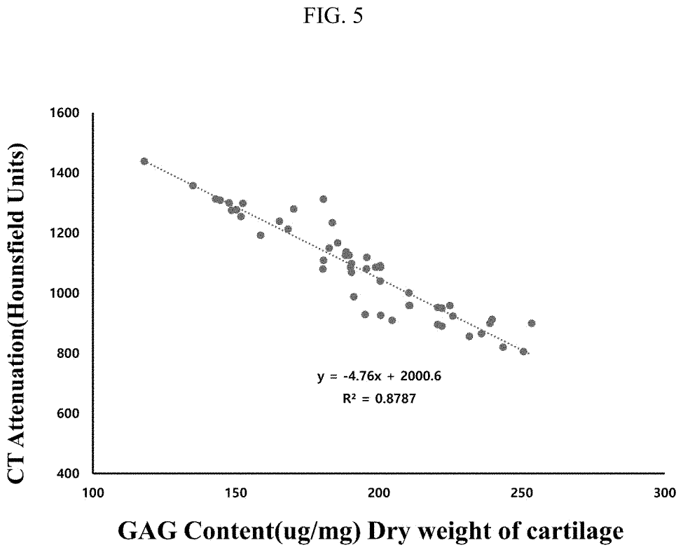

As a result, as shown in FIG. 5, by matching the analysis values of the biochemical assay and the analysis values of the Micro-CT assay in a graph, the formula “y=−4.76x+2000.6” [x: cartilage GAG content (μg/mg) dry weight, y: CT attenuation coefficient (HU)] was obtained. In this case, R² was 0.8787. With this formula, it is possible to derive the GAG content of a cartilage graft from the Micro-CT analytical value.

### Example 4: Micro-CT and Safranin-O Staining Assay of Cartilage Grafts

Micro-CT assay was performed on human cartilage tissue samples which were treated with a contrast medium diluted to a concentration of 60% (v/v) for each age according to Example 2, followed by histopathological assay of the same samples to qualitatively compare trends in GAG expression distribution. In the histological assay, the cartilage tissues were treated with 4% formalin for 7 days and then with 5% nitric acid solution for 24 hours for removal of calculus. Tissue blocks were then prepared via tissue processing and embedding, and 4 μm-thick slides were prepared using a Microtome instrument. Finally, Safranin-O staining, which is a GAG-specific tissue staining method, was performed to analyze a GAG expression distribution.

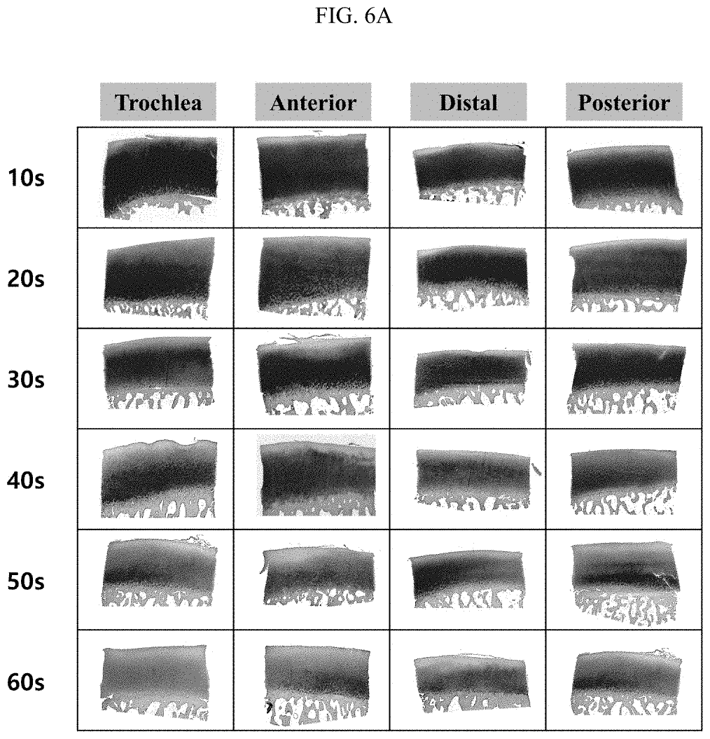

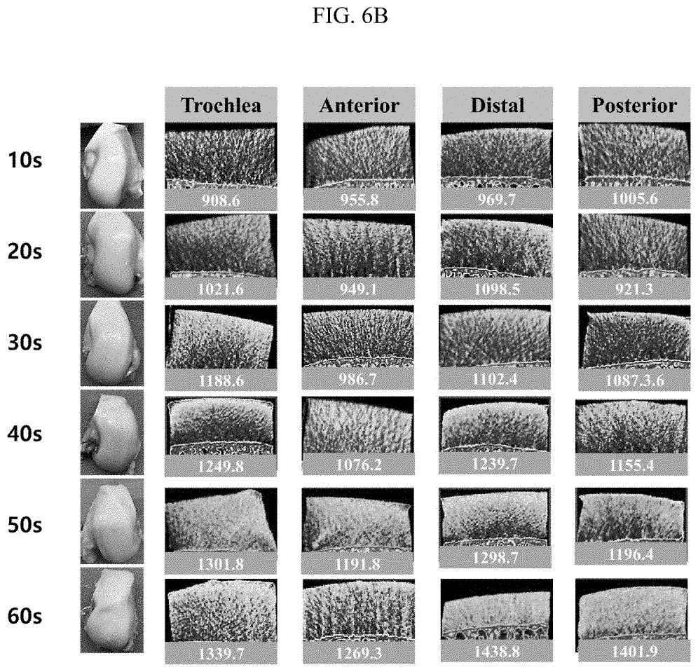

After the Safranin-O staining, the distribution of GAG expression was qualitatively assessed for each knee joint femoral cartilage site and for each age group as illustrated in FIG. 6A, and the image and optical density for each group were quantified through the Micro-CT assay as illustrated in FIG. 6B.

### Example 5: GAG Content Analysis of Rabbit Allogeneic Osteochondral Grafts

New Zealand rabbits (8 weeks old, 3-3.5 kg) were used in this study. After collecting osteochondral grafts from the rabbit trochlea using a biopsy punch with a diameter of 3 mm, the collected osteochondral grafts were treated with chondroitinase ABC (Sigma, C3667), which is a specific degrading enzyme for chondroitin sulfate, which is a major component of GAG, at a concentration of 1 unit/ml for 2, 4, and 8 hours. Then, changes in GAG content were evaluated through biochemical assay, Micro-CT assay, and histological assay.

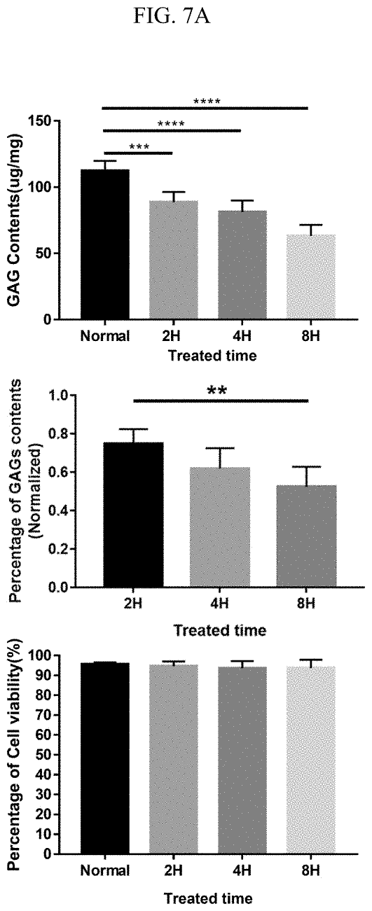

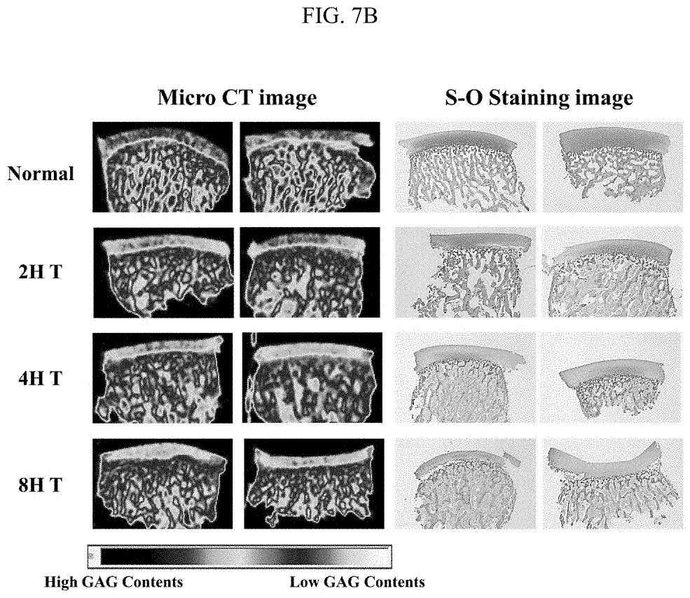

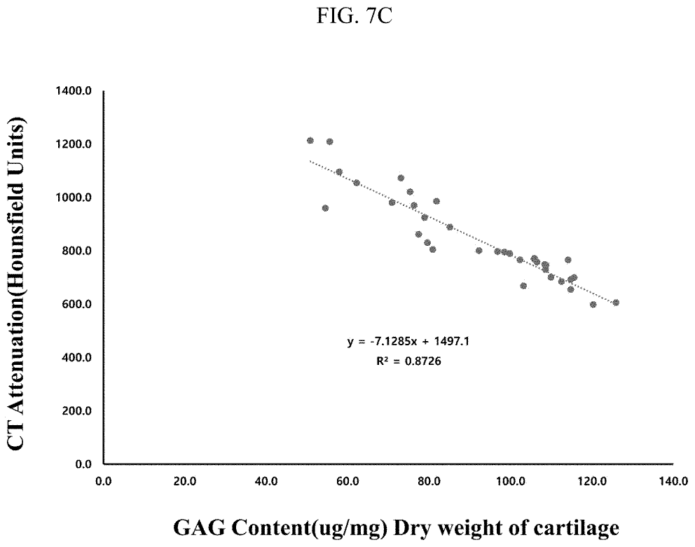

As a result, there were no significant changes in cell survival in cartilage tissue in each group over time of chondroitinase ABC treatment as shown in FIG. 7A.

Referring to FIGS. 7B and 7C, the correlation between the results derived by the Micro-CT assay and the results derived by the biochemical assay were formulated, and the formula “y=−7.1285x+1497.1 [x: cartilage GAG content (μg/mg) dry weight, y: CT attenuation coefficient (HU)]” was obtained. In this case, R² was 0.8726. With this formula, it is possible to derive the GAG content of a rabbit cartilage graft from the Micro-CT analytical value.

### Example 6: Evaluation of Effectiveness of Allogeneic Osteochondral Grafts According to GAG Content in Rabbit Model

A 3-mm osteochondral graft was harvested with a biopsy punch from the donor rabbit Trochlea site in a rabbit animal model. Next, a small amount of cartilage tissue is collected again from the vicinity of the site where the osteochondral graft was harvested, and the GAG content of the normal donor rabbit cartilage tissue was measured by the biochemical assay described in Example 2. The collected osteochondral grafts were classified into a 2-hour treatment group, a 4-hour treatment group, and an 8-hour treatment group according to the time of chondroitinase treatment and treated with chondroitinase for the determined periods according to the method described Example 5. Micro-CT assay was performed, and the HU values were quantitatively measured for each group. Next, allogeneic osteochondral transplantation was performed on the same site of each recipient rabbit with the prepared osteochondral grafts that were treated for different periods of time. After 4 weeks, the recipient rabbits with allogeneic osteochondral transplantation were sacrificed, and the therapeutic efficacy of cartilage regeneration was evaluated through visual evaluation, micro-CT assay, and histological assay.

As a result, referring to Table 1, it was confirmed that the GAG contents of normal cartilage tissue before the chondroitinase treatment of each group were 119.6±10.25, 125.8±8.25, and 116.2±12.81 μg/mg, respectively, and the Micro-CT HU values according to the chondroitinase enzyme treatment time for each group were 848.3±20.36 (2H), 922.5±28.41 (4H), and 1066.7±77.1 (8H).

TABLE 1

| Treatment | GAG Contents(Before | [symbol] Units(After | | GAG Contents(After | Percentage of |
|---|---|---|---|---|---|
| time | treatment) | treatment | → | treatment | GAG contents(%) |
| 2H Group | 119.6 ± 10.25 | 848.3 ± 20.36 | | 90.8 ± 2.21 | 75.4 ± 2.5% |
| 4H Group | 125.8 ± 8.25 | 922.5 ± 28.41 | | 20.6 ± 4.55 | 63.0 ± 4.8% |
| 8H Group | 116.2 ± 12.81 | 1066.7 ± 77.1 | | 60.3 ± 13.61 | 52.1 ± 8.4% |

[symbol]

indicates data missing or illegible when filed

The GAG contents after the enzyme treatment of the osteochondral grafts were calculated by inputting the CT HU values shown in Table 1 into the correlation formula described in Example 5. The GAG contents were 90.8±2.21 (2H), 20.6±4.55 (4H), 60.3±13.61 μg/mg (8H), respectively.

Next, the change in GAG content before and after chondroitinase enzyme treatment was 75.4±2.5 (2H), 63±4.8% (4H), 52.1±8.4% (8H), as a percentage, and it was confirmed that the GAG content decreased with chondroitinase treatment time.

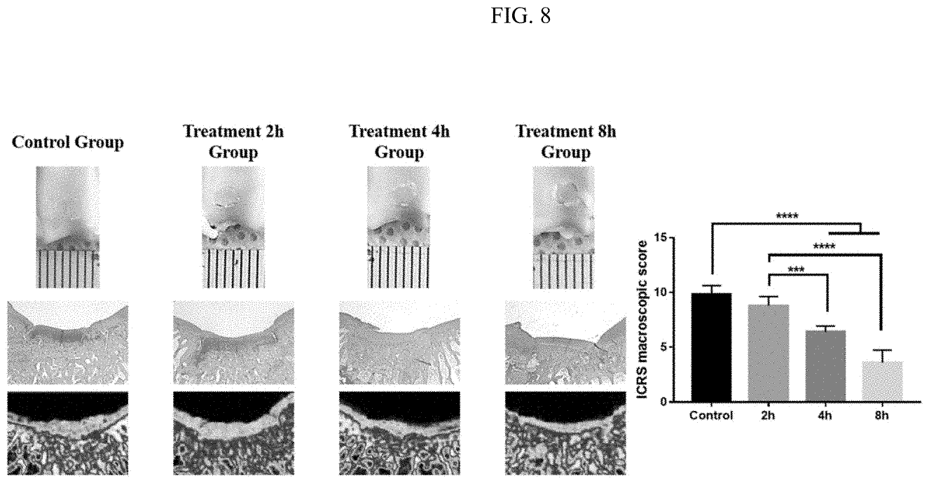

FIG. 8 shows the evaluation results of the effectiveness of cartilage tissue regeneration evaluated through appearance images, micro-CT assay, and histological assay of the samples after 4 weeks of allogeneic osteochondral transplantation, according to chondroitinase treatment times. Referring to FIG. 8, the appearance images of the normal control group and the chondroitinase 2-hour treatment group show that each graft is well fused with the surrounding tissue and exhibits a smooth surface, and an equilibrated morphology is observed. On the other hand, in the cases of the chondroitinase 4-hour treatment group and the chondroitinase 8-hour treatment group, each graft exhibits surface collapse, and the gap between the graft and the surrounding tissue is observed. This indicates that the donated osteochondral graft is not well fused with the surrounding tissue.

Next, the results of the Micro-CT and histological assays show that in the case of the chondroitinase 2-hour treatment group, the GAG content of cartilage graft tissue is maintained at a level similar to that of the normal control group, fusion with the surrounding tissue is achieved, and tissue regeneration is well induced without cartilage collapse in the subchondral bone.

On the other hand, in the cases of the chondroitinase 4-hour treatment group and the chondroitinase 8-hour treatment group, the grafts have an irregular surface, the GAG contents are significantly reduced, and a collapsed form of the subchondral bone is observed. Finally, when the healing of osteochondral tissue was quantitatively evaluated through ICRS evaluation, no statistical significance was observed between the normal control group and the chondroitinase 2-hour treatment group. On the other hand, it was confirmed that the ICRS scores of the chondroitinase 4-hour treatment group and the chondroitinase 8-hour treatment group were significantly lower than those of the other two groups. In conclusion, no significant changes were observed in the regeneration effectiveness of cartilage grafts when the GAG content was maintained in a level of 70% or higher compared to the normal cartilage. However, when the GAG content was reduced to 70% or below, it was confirmed that the effectiveness of cartilage regeneration after transplantation was reduced.

As described above, a specific part of the present disclosure has been described in detail, and those who ordinarily skilled in the art will appreciate that the specific description is only a preferred embodiment and the scope of the present disclosure is not limited by the specific description. Thus, the substantial scope of the present disclosure will be defined by the appended claims and their equivalents.

What is claimed is:

1. A method of analyzing quality of an osteochondral graft to use osteochondral tissue isolated from an individual as a graft for osteochondral transplantation, the method comprising analyzing a glycosaminoglycans (GAGS) content of the osteochondral tissue by using a Micro-CT assay.

2. The method of claim 1, wherein the Micro-CT assay is performed by diluting an iodine-based contrast medium with a phosphate buffer to a concentration range of 50% (v/v) to 60% (v/v) and treating the osteochondral tissue for 12 to 16 hours.

3. The method of claim 1, wherein the method determines the GAG content by converting a CT attenuation coefficient (Hounsfield unit, HU) value of Micro-CT and uses the determined GAG content as a criterion for determination of osteochondral graft quality.

4. The method of claim 1, wherein the iodine-based contrast medium is selected from the group consisting of Iohexol, Nucleosabrix, and Ioxaglate.

5. The method of claim 1, wherein when an osteochondral graft having a GAG content of 70% or higher is determined to be a suitable graft for osteochondral transplantation.

6. The method according to claim 1, wherein the osteochondral tissue is human- or rabbit-derived osteochondral tissue.

7. An osteochondral graft quality analysis method of calculating a glycosaminoglycan (GAG) content using a formula that reflects a correlation between a GAG content and a CT attenuation coefficient (HU) value of Micro-CT.

8. The method of claim 7, wherein the method calculates the GAG content of human osteochondral tissue using Formula 1:
y=−4.76x+2000.6 [Formula 1]
In Formula 1, x is the osteochondral GAG content (μg/mg) based on dry weight, and y is the CT attenuation coefficient (HU).

9. The method of claim 7, wherein the method calculates the GAG content of rabbit osteochondral tissue using Formula 2:
y=−7.1285x+1497.1 [Formula 2]
In Formula 2, x is the osteochondral GAG content (μg/mg) based on dry weight, and y is the CT attenuation coefficient (HU).
